(19) United States
(12) Patent Application Publication
(10) Pub. No.: US 2007/0264316 A1
(43) Pub. Date: Nov. 15, 2007

(54) Patch Activated In Use

(76) Inventors: Hirotoshi Adachi, Ibaraki (JP); Naruhito Higo, Ibaraki (JP); Seiji Tokumoto, Ibaraki (JP)

Correspondence Address: TOWNSEND & BANTA;c/o PORTFOLIO IP, PO BOX 52050, MINNEAPOLIS, MN, 55402, US

(21) Appl. No.: 10/584,172

(22) PCT Filed: Dec. 27, 2004
(86) PCT No.: PCT/JP04/19575

§ 371 Date: Mar. 21, 2007

(30) Foreign Application Priority Data
Dec. 26, 2003 (JP) 2003-434846

(51) Int. Cl. A61F 13/02

(52) U.S. Cl. 424/448

(57) ABSTRACT

Provided a patch activated in use capable of uniformly activating a drug by supplying a solution when used.

The patch activated in use comprises an absorber 11 containing a dry drug 10 and formed of a material capable of absorbing a liquid, a wall material 13 arranged around the absorber 11 and having an adhesive layer 12 on the lower surface thereof, a support 15 arranged on the absorber 11 and the wall material 13 and having an opening 14 at the center, a diaphragm 20 arranged on the support 15, and a dissolution liquid reservoir 18 arranged on the diaphragm 20, holding a dissolution liquid dissolving the drug in a space with the diaphragm 20 and having a protruding portion 17 which breaks the diaphragm 20 by pressure. The protruding portion 17 has a liner top end portion and arranged in contact or in the proximity with the diaphragm 20. A liner 19 is detachably provided on the lower surface of the absorber 11 and adhesive layer 12.

### TECHNICAL FIELD

The present invention relates to a patch activated in use, and more particularly, to a patch activated in use that is used after activation of a drug by supplying a dissolution liquid immediately before use.

### BACKGROUND ART

When a patch containing a chemically labile drug is manufactured by a conventional method, the stability of the drug is generally ensured by studying the composition (formulation) of the preparation. However, in some cases, sufficient stability of a drug may not be obtained by the study of formulation depending upon the nature of the drug.

Then, as a technique for stabilizing a drug, there is known that the drug is previously made in a solution form, stored in a container, and supplied to a drug permeable layer by breaking the container when used. For example, Patent Document 1 discloses a dermatologic patch for external use having a flowable drug enclosed in a blister portion. The dermatologic patch is formed of an adhesive sheet and a release sheet. The adhesive sheet has a blister portion formed of a blister and a drug coating layer, which seals the lower surface of the blister, and a drug permeating layer formed under the drug coating layer. When used, the user presses the blister by a finger, thereby breaking the drug coating layer to transfer a fresh drug into the drug permeating layer. The dermatologic patch has a concave or convex protrusion provided to the blister or enclosed within the blister portion. The protrusion functions not only to break the drug coating layer easily and without fail but also to give direct acupressure or stimulate an acupressure point.

Patent Document 1: Japanese Patent Application Laid-Open No. 9-124468

On the other hand, another patch is known, which is used by supplying a liquid to a dry drug. For example, Patent Document 2 discloses an adhesive bandage having a piece of gauze at the center of an adhesive tape. The adhesive bandage is formed by placing a piece of sterilized dry gauze containing a disinfectant or a therapeutic drug at the center of the adhesive surface of the adhesive tape, adhering an immobilizing end to the adhesive surface of the adhesive tape, providing an adhesive surface cover so as to cover the gauze, and providing a liquid pouch covered by a easy-to-break thin film material, which is less stronger than the adhesive surface cover, to the portion facing the gauze of the adhesive surface cover. In the pouch, sterilizing liquid or sterilized distilled water is contained. In using, when the user strongly presses the adhesive cover to break the thin film, the gauze gets wet with a disinfectant solution or sterilized distilled water contained in the liquid pouch to effectively produce the medicinal effect of the drug.

Patent Document 2: Japanese Utility Model Publication No. 54-23197

### DISCLOSURE OF THE INVENTION

Problems to be Solved by the Invention

These conventional techniques have problems. In the technique disclosed in Patent Document 1, a drug solution is supplied to a drug permeable layer by breaking a container when used. This technique is applicable to a drug that can be stored stably in a solution, but unfavorable to other drugs that are unstable in a solution, in view of stability. Also, in the technique disclosed in Patent Document 2, a liquid is supplied to a dry drug, when used. In this case, the liquid pouch is covered with an easy-to-break thin material, so that the portion of the pouch that is broken when the adhesive cover is pressed is not determined. For example, if the thin film of the pouch breaks at its end, the liquid may not be spread all over the gauze.

Thus, an object of the present invention is to provide a patch activated in use with which a drug can be activated uniformly by supplying a solution, when used.

Means for Solving the Problems

The aforementioned object can be attained by a patch activated in use comprising: an absorber containing a dry drug and formed of a material capable of absorbing a liquid; a wall material arranged around the absorber and having an adhesive layer on the lower surface thereof; a support arranged on the absorber and the wall material and having an opening at the center; a diaphragm arranged on the support; and a dissolution liquid reservoir arranged on the diaphragm, holding a dissolution liquid dissolving the drug in a space with the diaphragm, and having a protruding portion which breaks the diaphragm by pressure. The patch may further have a solution permeable film on the lower surface of the absorber. Also, on the lower surface of the absorber and the adhesive layer, a liner having a concaved portion facing the absorber may be provided.

Another patch activated in use according to the present invention comprising: a drug containing layer containing a dry drug; an absorber arranged on the drug containing layer and formed of a material capable of absorbing a liquid; a wall material arranged around the absorber and having an adhesive layer on the lower surface thereof; a support arranged on the absorber and the wall material and having an opening at the center; a diaphragm arranged on the support; and a dissolution liquid reservoir arranged on the diaphragm, holding a dissolution liquid dissolving the drug in a space with the diaphragm, and having a protruding portion which breaks the diaphragm by pressure. In the patch, a liner having a concaved portion facing the drug containing layer may be provided on the lower surface of the drug containing layer and the adhesive layer.

Another patch activated in use according to the present invention comprising: a support; an absorber arranged on the support, containing a dry drug, and formed of a material capable of absorbing a liquid; a wall material arranged on the support and around the absorber and having an adhesive layer on the upper surface thereof; a liner arranged on the absorber and the adhesive layer and having an opening at the center,
a diaphragm arranged on the liner; and a dissolution liquid reservoir arranged on the diaphragm, holding a dissolution liquid dissolving the drug in a space with the diaphragm, and having a protruding portion which breaks the diaphragm by pressure. In the patch, a solution permeating film can be provided on the upper surface of the absorber.

Another patch activated in use according to the present invention comprising: a support; an absorber arranged on the support and formed of a material capable of absorbing a liquid; a wall material arranged on the support and around the absorber and having an adhesive layer on the upper surface thereof; a drug containing layer arranged on the absorber and containing a dry drug; a liner arranged on the drug containing layer and the adhesive layer and having an opening at the center; a diaphragm arranged on the liner; and a dissolution liquid reservoir arranged on the diaphragm, holding a dissolution liquid dissolving the drug in a space with the diaphragm, and having a protruding portion which breaks the diaphragm by pressure.

In the patch, a portion of the diaphragm in contact with the dissolution liquid may have an oval shape and the protruding portion of the dissolution liquid reservoir may have a linear top end portion extending along the longitudinal axis of the oval. In this case, assuming that the length of the linear top end portion is represented by L1 and the length of the longitudinal axis of the portion of the diaphragm in contact with the dissolution liquid is represented by L2, the following relationship is preferably satisfied.
0.1×L2≦L1≦0.5×L2

Furthermore, a portion of the diaphragm in contact with the dissolution liquid may have a circular shape and the protruding portion of the dissolution liquid reservoir may have a cruciform top end portion. In this case, assuming that the lengths of both bars of the cruciform top end portion are represented by L10 and L11, respectively, and the diameter of the portion of the diaphragm in contact with the dissolution liquid is represented by L2, the following relationship is preferably satisfied.
0.1×L2≦L10≦0.5×L2 and/or 0.1×L2≦L11≦0.5×L2

Furthermore, the portion of the support around the opening is preferably depressed toward the absorber compared to the other portion. Moreover, the support inclines from the peripheral portion toward the opening while depressed toward the absorber. Similarly, the portion of the liner around the opening is depressed toward the absorber compared to the other portion. Also, the liner inclines from the peripheral portion toward the opening with respect to the absorber.

The dissolution liquid reservoir is preferably formed by subjecting a sheet material to mold processing and the sheet material preferably has a water vapor permeability of 0.22 g/m²·24 hr or less. The sheet material preferably has a thickness of about 250 μm to about 350 μm. The sheet material may contain, for example, a cyclic polyolefin copolymer film, and preferably, a laminate film of a cyclic polyolefin copolymer film and a polyolefin film. The sheet material may include a fluorocarbon resin film, and preferably a laminate film of a fluorocarbon resin film and a polyolefin film. Furthermore, the diaphragm may be an aluminium foil.

### ADVANTAGES OF THE INVENTION

According to the present invention, it is possible to obtain a patch activated in use with which a drug can be activated uniformly by supplying a dissolution liquid, when used. When the dissolution liquid is supplied in the manner according to the present invention, the concentration of a drug can be virtually equalized at all parts of the patch. Furthermore, the solution of the dissolution liquid reservoir can quickly flow when used, so that the amount of solution remaining in the dissolution liquid reservoir can be reduced.

### BRIEF DESCRIPTION OF THE DRAWINGS

FIG. 1 (a) is a plan view of a structure of a patch activated in use according to the present invention and FIG. 1(b) is a sectional view taken along the line X-X of the FIG. 1 (a);

FIG. 2 is a sectional view of another structure of the patch activated in use according to the present invention;

FIG. 3 is a sectional view of another structure of the patch activated in use according to the present invention;

FIG. 4 is a sectional view of another structure of the patch activated in use according to the present invention;

FIG. 5 is a sectional view of another structure of the patch activated in use according to the present invention;

FIG. 6 is a sectional view of another structure of the patch activated in use according to the present invention;

FIG. 7 is a sectional view of still another structure of the patch activated in use according to the present invention;

FIG. 8 is a sectional view of still another structure of the patch activated in use according to the present invention;

FIG. 9 (a) is a plan view of a structure of a dissolution liquid reservoir for use in a patch activated in use according to the present invention, FIG. 9(b) is a sectional view taken along the line X-X of the FIG. 9 (a), and FIG. 9(c) is a sectional view taken along the line Y-Y of the FIG. 9(a);

FIG. 10 is a plan view of another structure of the dissolution liquid reservoir for use in a patch activated in use according to the present invention;

FIG. 11 is a sectional view showing a structure of a support or a liner according to the present invention; and

FIG. 12 is a sectional view showing another structure of a support or a liner according to the present invention.

### DESCRIPTION OF SYMBOLS

10, 40 Drug

11, 41 Absorber containing a dry drug

12, 42 Adhesive layer

13, 43 Wall material

14, 44 Opening

15, 45 Support

16, 46, 91 Dissolution liquid

17, 47, 94 Protruding portion

18, 48, 90 Dissolution liquid reservoir

19, 49, 79, 89 Liner

20, 50, 92 Diaphragm

21, 51 Solution permeable film

31, 61 Absorber containing no drug

32, 62 Drug containing layer

### BEST MODE FOR CARRYING OUT THE INVENTION

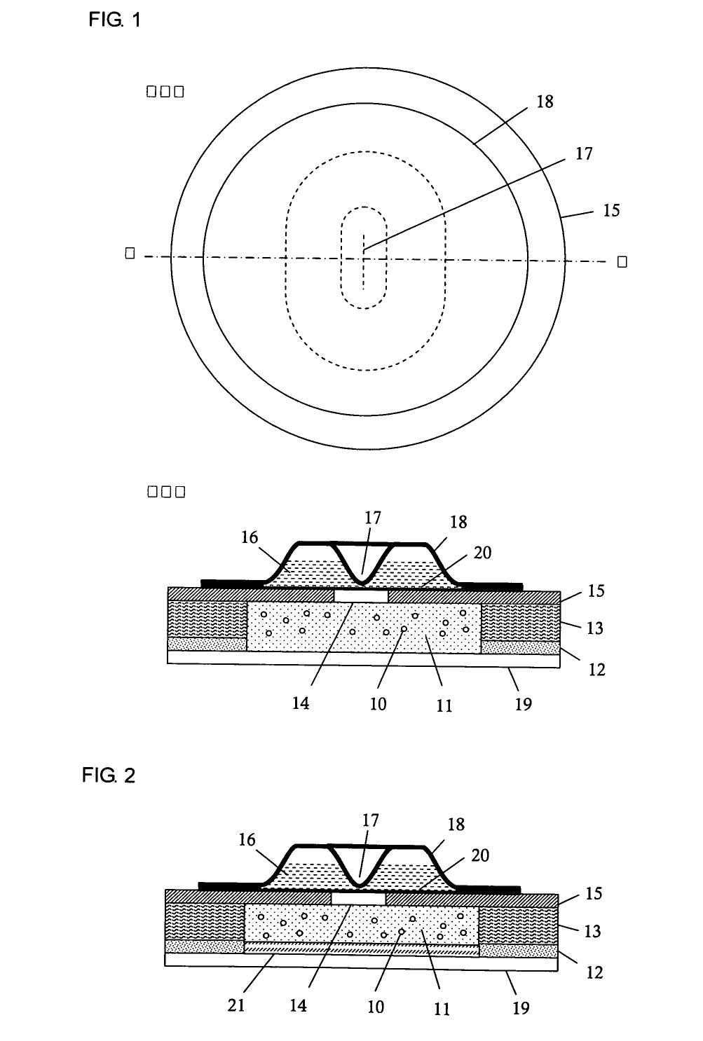

FIG. 1 (a) is a plan view of a structure of a patch activated in use according to the present invention and FIG. 1(b) is a sectional view taken along the line X-X of the FIG. 1 (a). As shown in the figures, a patch activated in use of this embodiment comprises an absorber 11 containing a dry drug 10 and formed of a material capable of absorbing a liquid, a wall material 13 arranged around the absorber 11 and having an adhesive layer 12 on the lower surface thereof, a support 15 arranged on the absorber 11 and the wall material 13 and having an opening 14 at the center, a diaphragm 20 arranged on the support 15, and a dissolution liquid reservoir 18 arranged on the diaphragm 20 holding a dissolution liquid dissolving the drug in a space with the diaphragm 20, and having a protruding portion 17 which breaks the diaphragm 20 by pressure. The protruding portion 17 has a linear top end portion as shown in the figure, for example, and arranged in contact or in the proximity of the diaphragm 20. On the lower surface of the absorbent 11 and the adhesive layer 12, a liner 19 is detachably attached. In this embodiment, the dissolution liquid reservoir 18 and the diaphragm 20 may be separately or integrally formed. A dissolution liquid container is formed of the dissolution liquid reservoir 18 and the diaphragm 20 in combination. The shape of the opening 14 of the support is not particularly limited. Any shape may be employed as long as it can supply the solution uniformly to the absorber 11, for example, a circular shape is preferable. In this case, the dimension of the opening varies depending upon the size of the absorber 11, for example, the opening has a diameter of 2 to 10 mm, and preferably 4 to 8 mm. Note that the support 15 may be omitted and instead, the diaphragm may substitute the function of the support. In this case, an opening is not previously provided. The opening comes to be provided by a protruding portion when used.

In using, when the user presses the upper surface of the dissolution liquid reservoir 18 or the protruding portion 17, the protruding portion 17 breaks the diaphragm 20. At this time, the diaphragm 20 greatly breaks along the liner top end portion, with the result that the solution in the dissolution liquid reservoir 18 flows through the opening 14 of the support 15 to the absorber 11. By virtue of the solution, the absorber 11 gets wet to activate a drug 10 uniformly. Thereafter, the liner 19 is removed and the patch of the present invention is applied to the skin. In this manner, an activated drug is permeated into the skin. In this embodiment, the dissolution liquid reservoir 18 is fixed to the patch itself. Therefore, the patch may be applied without waiting until the dissolution liquid reservoir becomes empty. This is because even if the dissolution liquid remains in the dissolution liquid reservoir 18 when used, it is gradually supplied to the absorber 11.

In the patch according to the present invention, the following substances may be used in individual portions. As a drug, any type of drug may be used depending upon the therapeutic purpose. As long as a compound has a pharmacological activity, it is not particularly limited by the type of drug, type of a salt, and application of drug. Example of such a compound may include antibiotic, antifungal drug, antitumor drug, cardiac stimulant , antiarrhythmic, vasodilator, antihypertensive drug, diuretic drug, antihypertensive/diuretic drug, drug for circulatory organs, antiplatelet drug, hemostatic drug, hypolipidemic drug, antipyretic/analgesic/antiinflammatory drug, antirheumatic drug, muscle relaxant, antitussive and expectorant, antiulcer drug, sedative drug, antiepileptic drug, antidepressant drug, antiallergic drug, diabetic diabetes mellitus therapeutic drug, antituberculous drug, hormone drug, narcotic antagonist, bone absorption suppressing drug, neovascularization suppressing drug, and local narcotic drug.

Examples of the antibiotic include gentamicin sulphate, lividomycin, sisomicin sulfate, tetracycline hydrochloride, ampicillin, cefalotin sodium, cefotiam hydrochloride, cefazolin sodium, thienamycin, sulfazecin, streptomycin sulfate, kanamycin sulfate, rifampicin, vancomycin hydrochloride, ofloxacin, and cefoselis sulfate.

Examples of the antifungal agent include amphotericin B, itraconazole, fluconazole, miconazole, 2-[(1R,2R)-2-(2,4-difluoro phenyl-2-hydroxy-1-methyl-3-(1H-1,2,4-triazol-1-yl)propox y)-4-[4-2,2,3,3-tetrafluoropropoxy]phenyl]-3(2H,4H)-1,2,4-triazolon.

Examples of the antitumor agent include bleomycin hydrochloride, tegafur, actinomycin D, mitomycin C, adriamycin, fluorouracil, 6-mercaptopurine, cytarabine, procarbazine, doxorubicin hydrochloride, methotrexate, and tamoxifen citrate.

Examples of the antituberculous agent include streptomycin sulfate, kanamycin sulfate, isoniazid, ethambutol hydrochloride, and pyrazinamide.

Examples of the cardiac stimulant include trance-bio Kiso camphor, teofirol, dopamine hydrochloride, dobutamine hydrochloride, and ubidecarenone.

Examples of the antiarrhythmic include propranolol hydrochloride, oxyprenolol, procainamide hydrochloride, lidocaine, phenytoin, metoprolol tartrate, verapamil hydrochloride, and diltiazem hydrochloride.

Examples of the vasodilator include oxyfedrine hydrochloride, tolazoline hydrochloride, bamethan sulfate, nicardipine hydrochloride, verapamil hydrochloride, and papaverine hydrochloride.

Examples of the antihypertensive drug include hydralazine hydrochloride, budralazine, prazosinhydrochloride, doxazosin mesilate, carteolol hydrochloride, clonidine hydrochloride, enalapril maleate, captopril, delapril hydrochloride, manidipine hydrochloride, pinacidil, minoxidil, losartan, candesartancilexetil, valsartan, telmisartan, andirbesartan.

Examples of the diuretic drug include acetazolamide, methazolamide, chlorothiazide, furosemide, triamterene, amiloride, and aminometrozine.

Examples of the antihypertensive/diuretic drug include include pentolinium, and hexamethonium bromide.

Examples of the drug for circulatory organs include alprostadil, alprostadil alfadex, limaprost, ozagrel sodium, clopidogrel sulfate, prostacyclin, beraprost, ciprostene, ailoprost,ataprost,clinprost, ethylicosapentate, etilefrine hydrochloride, dihydroergotamine mesylate, pamicogrel, tranilast, probucol, candesartan cilexetil, sodium citrate, heparin, low molecular weight heparin, nifedipine, efonidipine hydrochloride, diltiazem hydrochloride, and tranilast.

Example of the antiplatelet drug include ticlopidine, satigrel, limaprost alfadex, clinprost, clopidogrel sulfate, sibrafiban, epitibatide, tirofiban hydrochloride, sarpogrelate hydrochloride, xemilofiban hydrochloride, orbofiban acetate, isbogrel, cilostazol, aspirin, and abciximab.

Examples of the hemostatic drug include epinephrine, menadione sodium bisulfite, acetomenaphthone, and tranexamic acid.

Examples of the hypolipidemic drug include sodium pravastatin, simvastatin, fuvastatin sodium, crivastatin, and atorvastatin.

Examples of the antipyresis/analgesiac/antiinflammatory drug include aspirin, sodium salicylate, sulpyrine, indomethacin, diclofenac sodium, loxoprofen sodium, felbinac, zaltoprofen, piroxicam, nimesulide, Meloxicam, celecoxib, tiaramide, emorfazone, buprenorphine, eptazocinehydrobromide, pentazocine, butorphanol tartrate, tramadol hydrochloride, Ketorolac, meperidine hydrochloride, morphine hydrochloride, morphine sulfate, hydro-morphine, fentanyl citrate, fentanyl, and mofezolac.

Examples of the antirheumatic drug include methotrexate hydrochloride, sodiumaurothiomalate, auranofin, bucillamine, D-penicillamine, actarit, lobenzarit, mizoribine, salazosulfapyridine, and tacrolimus hydrate.

Examples of the muscle relaxant include pridinol methane sulfonate, tubocurarine chloride, eperisone hydrochloride, tizanidine hydrochloride, chlorphenesin carbamate, tolperisone hydrochloride, dantrolene sodium, baclofen, and lanperisone hydrochloride.

Examples of the antitussive and expectorant include ephedrine hydrochloride, codeine phosphate, picoperidamine hydrochloride, ambroxol, bromhexinehydrochloride, salbutamol sulfate, tulobuterol hydrochloride, formoterol fumarate, azelastine hydrochloride, ketotifen fumarate, and picoperidamine.

Examples of the antiulcer drug include ornoprostil, cimetidine, famotidine, ranitidine hydrochloride, metoclopramide, omeprazole, and lansoprazole.

Examples of the sedative drug include chlorpromazine hydrochloride, atropine sulfate, and fluphenazine enanthate.

Examples of the antiepileptic drug include phenytoin sodium, and ethosuximide.

Examples of the antidepressant drug include amitriptyline hydrochloride, imipramine hydrochloride, clomipramine hydrochloride, desipramine hydrochloride, maprotiline hydrochloride, and phenelzine sulfate.

Examples of the antiallergic agent include diphenhydramine, hydrochloride, tripelennamine hydrochloride, clemizole, d-chlorpheniramine maleate, cyproheptadine hydrochloride, ketotifen fumarate, epinastine, and tacrolimus hydrate.

Examples of the diabetes mellitus therapeutic drug include glymidine zodium, glipizide, metformin, tolbutamide, chlorpropamide, glibenclamide, acetohexamide, midaglizole, glimepiride, senaglinide, repaglinide, and pioglitazone hydrochloride.

Examples of the antituberculous drug include streptomycin sulfate, kanamycin sulfate, isoniazid, ethambutol hydrochloride, and pyrazinamide.

Examples of the hormone drug include β-estradiol, testosteroneenanthate, prednisolonesuccinate, dexamethasone sodium phosphate, and methimazole.

Examples of the narcotic antagonist include levallorphan tartrate, nalorphine hydrochloride, protamine, and naloxone.

Examples of the bone absorption suppressing drug include sulfur-containing alkyl aminomethylene bisphosphonate, raloxifene, aendronate sodium, Incadronate Disodium, tibolone, simadronate, risedronate, clodronate disodium, falecalcitriol, calcitriol, alpha calcitriol, didronel sodium, ipriflavone, and minodronic acid.

Examples of the neovascularization suppressing drug include arterialization depression steroid [see Science Vol. 221, page 719 (1983)] and fumagillol derivatives [such as monochloroacetylcarbamoylfumagillol, O-dichloroacetylcarbamoylfumagillol (see European Patent application Nos. 357,061, 359036, 386667 and 415294)].

Examples of the local anesthetic drug include lidocaine hydrochloride, tetracaine hydrochloride, procaine hydrochloride, benzocaine hydrochloride, etidocaine hydrochloride, prilocaine hydrochloride, dibucaine hydrochloride, bupivacaine hydrochloride, cocaine hydrochloride, ethylaminobenzoate, orthocainehydrochloride, oxethazaine hydrochloride, and mepivacaine hydrochloride.

In addition to drugs, a drug dissolution rate regulator, an additive(s) for stabilization, an adsorption inhibitor may be added.

As the absorber, a material capable of absorbing a liquid satisfactorily is selected. Examples of such a material include polyester (a polyethylene terephthalate) , polysaccharides and cellulose derivatives (rayon, cotton), polyamide (nylon), porous materials such as a nonwoven fabric, cloth, gauze and sponge, and hydrophilic polymers (agar, agarose, alginic acid, xanthan gum, guar gum, dextran, dextrin, Pullulan, chitosan, gelatine, carboxyvinyl polymer, polyacrylate, carboxymethyl cellulose salt, polyoxyalkylene, polyvinyl alcohol, polyvinylpyrrolidone, and polyacrylamide), and ion exchange resin (amberlite, diaion, cholestyramine); however, nonwoven fabric such as fabric principally made of rayon is preferably used, for example.

As the wall material, a water-impermeable material is selected such as foaming polyolefin (PE, PP), foaming polyurethane, foaming polystyrene, foaming gum (e.g. polybutylene), foaming EVA, and foaming PVC; however, foaming polyolefin is preferably used.

As the adhesive layer, mention may be made of natural rubber, styrene-isoprene-styrene block copolymer, styrene-butadien gum, styrene-isoprene gum, polyisobutylene, polyisoprene, polyacrylate, and silicon gum; however, polyacrylate is preferably used, for example.

As the supporting material, a water-impermeable material is selected such as polyolefin, polyurethane, polystyrene, gum, EVA, PVC, and PET.

The dissolution liquid reservoir may be formed of a molded sheet obtained by molding a sheet material such as PET, PVC, PVDC, PP, PE, polystyrene, cyclic polyolefin (COC), Al and a laminate body of these in the dome form and forming a convex protruding portion within the dome, or a sheet having a high barrier property (such as PCTFE/PP series, PCTFE/PVC series, cyclic polyolefin/PP series), and Al deposition sheet and SiO₂ deposition sheet. When the user presses the convex protruding portion of the dissolution liquid reservoir, the diaphragm or at least one of the stacked structure of the diaphragm and the support is broken. When the convex protruding portion has a conical form, the shape of the broken portion is a point. In this case, the permeation of the solution to the absorber is poor. Therefore, the breaking portion (top end portion of the protruding portion) of the convex form is preferably liner or planar. The material of the dissolution liquid reservoir may be PCTFE(-CF2-CFC1-)ₙpoly (chloro-trifluoroethylene), or a COC cyclic polyolefin copolymer. The thickness of the sheet is, for example, 100 to 500 μm. As the material for the dissolution liquid reservoir, for example, PP, PP/COC/PP, and PCTFE/PP series may be used.

As the diaphragm (the film to be broken by the protruding portion), Al, PP, PE and a laminate body of them may be mentioned. When Al foil is used, it may be preferably coated in order to prevent corrosion, as needed. The thickness of the diaphragm is, for example, 5 to 100 μm in the case of Al, 15 to 50 μm in the case of PP and PE.

Example of the solution include water, alcohols, polyhydric alcohols, surfactants, saccharides, pH modifiers (organic and inorganic acids/base), salines, water soluble polymers, solubilizing agents, absorption accelerator, fats and oils, and preservatives. Preferable examples include purified water, glycerin, methylparaben (propylparapen, propylene glycol). Example of the liner include PET, PEN, PP, PE, paper, Al, and a laminate of these. Preferably, PET is used. In addition, it is preferable to apply mold-releasing treatment such as silicon treatment to the surface of the liner. Furthermore, the liner may be processed in a concave form so as not to come in contact with a member containing a drug.

FIG. 2 is a sectional view showing another structure of a patch activated in use according to the present invention. Difference of the patch of this embodiment from that of FIG. 1 resides in that a solution permeable film 21 is provided on the lower surface of the absorber 11 containing a drug. The other portions are the same as those of FIG. 1. The solution permeable film 21 is effective in holding the absorber and further provided as holding means when a powdery substance is contained.

As the solution permeable film, a porous film or ion exchange membrane may be used. Examples of the porous film include PE, PP, cellulose, cellulose acetate, PET, and nylon. Examples of the ion exchange membrane include a positive ion exchange membrane, a negative ion exchange membrane, and a composite charged membrane. Preferably a positive ion exchange membrane of a nylon series may be used. However, when nonwoven cloth is used as the absorber, the solution permeable film may not be used.

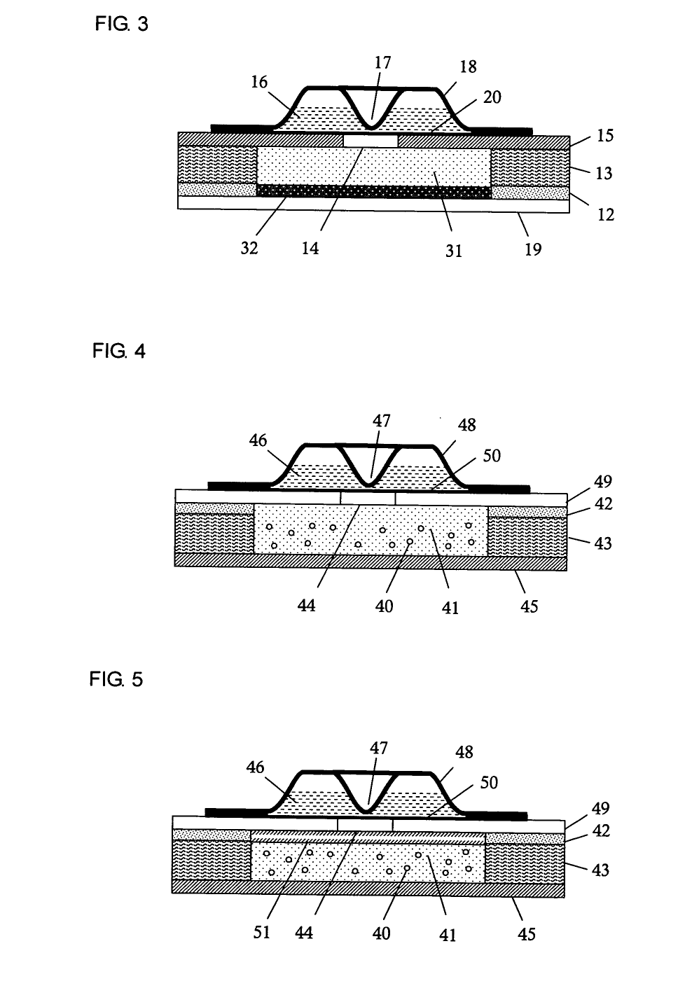

FIG. 3 is a sectional view of another structure of the patch activated in use according to the present invention. The patch of this embodiment is characterized in that the absorber 11 containing a drug of FIG. 1 is divided into two layers, an absorber 31 containing no drug and a drug containing layer 32 containing a drug. The other structural elements are the same as in FIG. 1. The reason why the absorber 31 containing no drug and the drug containing layer 32 are separately formed is that the drug is brought into contact with a living body at a high concentration to maximize the absorption of a drug.

As the drug containing layer, a porous film or ion exchange membrane containing a drug may be used. Examples of the porous film may include PE, PP, cellulose, cellulose acetate, PET, and nylon. Examples of the ion exchange membrane include a positive ion exchange membrane, negative ion exchange membrane, and an composite charged membrane. Preferably a positive ion exchange membrane of a nylon series may be used.

FIG. 4 is a sectional view of another structure of the patch activated in use according to the present invention. Unlike from those of FIGS. 1 to 3, when the patch of this embodiment is used, a dissolution liquid reservoir is removed. As shown in the figure, the patch of the embodiment comprises: a support 45, an absorbing material 41 arranged on the support 45, containing a dry drug 40, and formed of a liquid absorbable material, a wall material 43 arranged around the absorber 41 and on the support 45 and having an adhesive layer 42 on the upper surface, a liner 49 arranged on the absorber 41 and the adhesive layer 42 and having an opening 44 at the center, a diaphragm 50 arranged on the liner 49, and a dissolution liquid reservoir 48 arranged on the diaphragm 50, holding a solution for dissolving a drug in a space with the diaphragm 50, and having a protruding portion 47 for breaking the diaphragm 50 by pressure.

The protruding portion 47 is constructed in the same manner as in FIG. 1. The liner 49 is detachably attached to the adhesive layer 42. The shape and size of the opening 44 of the liner are the same as in FIG. 1.

In using, when the user presses the upper surface of the dissolution liquid reservoir 48, the protruding portion 47 breaks the diaphragm 50. When the diaphragm 50 is broken by the pressure of the protruding portion 47, the solution inside the dissolution liquid reservoir flows out through the opening 44 of the liner 49 to the absorber 41. The absorber 41 gets wet by the solution to activate a drug uniformly. Thereafter, the liner 49 is removed together with the dissolution liquid reservoir 48 and then the patch is applied to the skin. In this way, the activated drug permeates into the skin. In this embodiment, since the dissolution liquid reservoir 48 is removed together with the liner 49 from the main body of the patch when used, the patch is preferably applied after the solution completely flows out from the dissolution liquid reservoir 48.

FIG. 5 is a sectional view of another structure of the patch activated in use according to the present invention. The difference of the patch of this embodiment from that of FIG. 4 resides in that a solution permeable film 51 is provided on the upper surface of the absorber 41 containing a drug. The other structural elements are the same as in FIG. 4. The reason why the solution permeable film 51 is provided and the material of the film are the same as described in connection with FIG. 2.

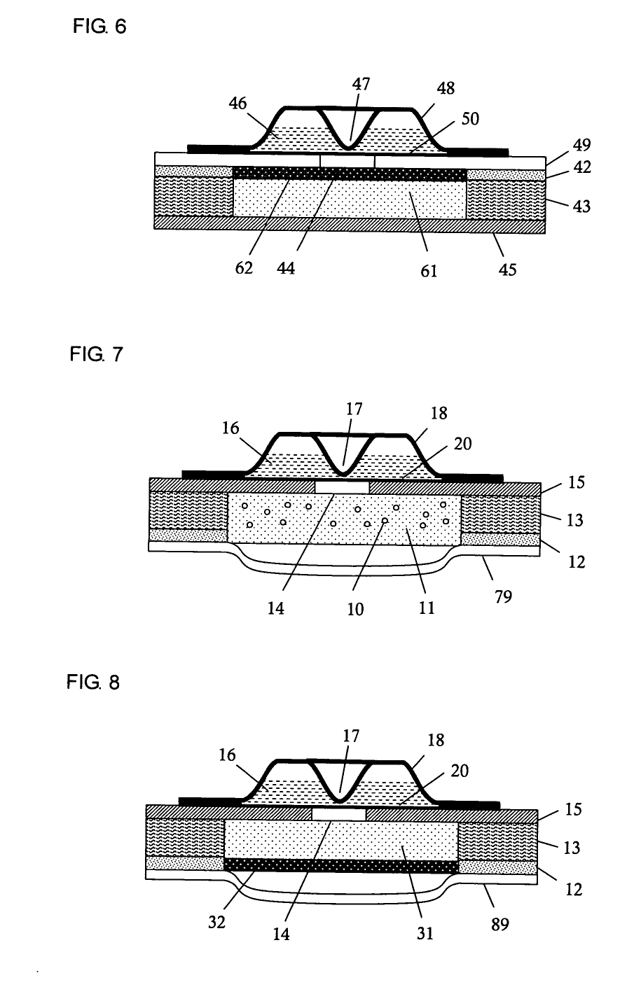

FIG. 6 is a sectional view of another structure of the patch activated in use according to the present invention. The patch of this embodiment is characterized in that the absorber 41 containing a drug in FIG. 4 is divided into two portions, an absorber 61 containing no drug and a drug containing layer 62 containing a drug. The other structural elements are the same as in FIG. 4. The reason why the absorber 61 and the drug containing layer 62 are separately formed and the material of the film are the same as described in connection with FIG. 3.

FIG. 7 is a sectional view of another structure of the patch activated in use according to the present invention. The difference of the patch of this embodiment from that of FIG. 1 resides in that a liner 79 having a concaved form facing the absorber is provided on the lower surface of the absorber and the adhesive layer. The other structural elements are the same as in FIG. 1. The reason why the liner 79 is processed in the concaved form is to prevent the liner from being in contact with the member containing a drug.

FIG. 8 is a sectional view of another structure of the patch activated in use according to the present invention. The difference of the patch of this embodiment from that of FIG. 3 resides in that a liner 89 having a concaved form facing the drug containing layer is provided on the lower surface of the drug containing layer and the adhesive layer. The other structural elements are the same as in FIG. 3. The reason why the liner 89 is processed in the concaved form is to prevent the liner from being in contact with the member containing a drug.

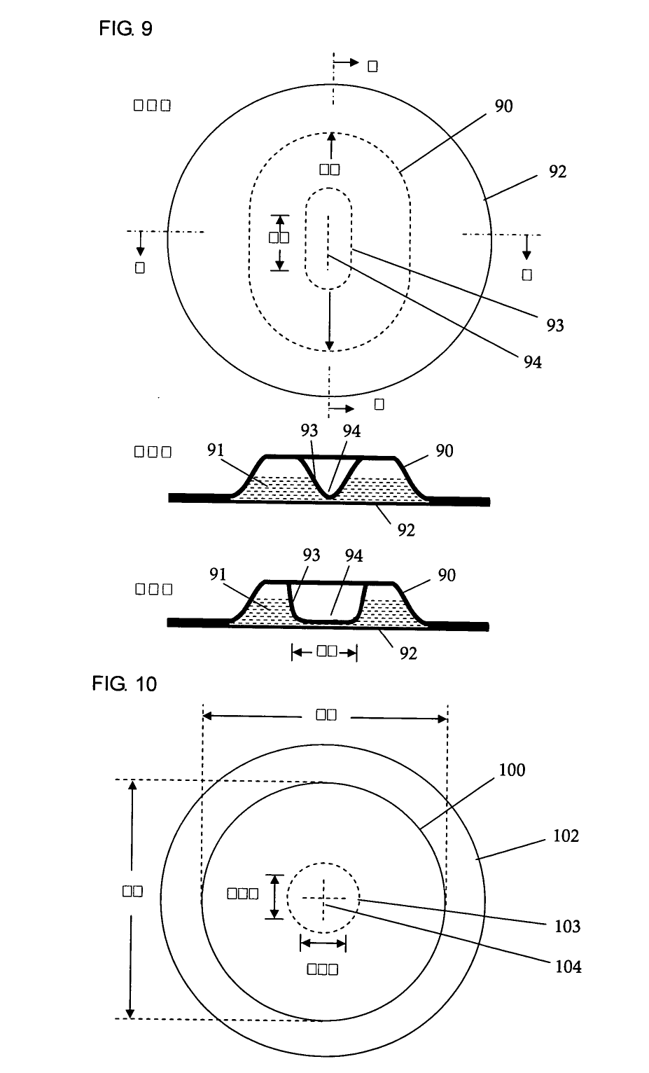

FIG. 9(a) is a plan view showing a structure of a dissolution liquid reservoir to be used in the patch activated in use according to the present invention, FIG. 9(b) is a sectional view taken along the line X-X of the FIG. 9(a), and FIG. 9(c) is a sectional view taken along the line Y-Y of the FIG. 9(a). In this embodiment, the structure is designed such that the solution of the dissolution liquid reservoir can be supplied to the absorber to reduce the remaining amount of solution as much as possible. A dissolution liquid reservoir 90 of this embodiment stores a solution 91 and has a protruding portion 93 for breaking a diaphragm 92 by pressure. As shown in the figure, the portion of the diaphragm 92 in contact with the solution has an oval shape and diaphragm 92 itself has a circular shape. The protruding portion 93 has a linear top end portion 94 extending along the longitudinal axis of an oval-shape portion of the diaphragm 92 in contact with the solution. Assuming that the length of the top end portion 94 is represented by L1, and the length of the longitudinal axis of the portion of the diaphragm 92 in contact with the solution is represented by L2, they are constructed so as to satisfy the following relationship:
0.1×L2≦L1≦0.5×L2.

If so, when the protruding portion 93 is pressed in use and the diaphragm 92 is greatly broken along the linear top end portion, the solution 91 flows out well, with the result that the remaining amount of solution can be reduced. In this embodiment, the top linear portion 94 is arranged at a distance from the diaphragm 92; however, they may be arranged in contact with each other.

FIG. 10 is a plan view showing another structure of the dissolution liquid reservoir to be used in a patch activated in use according to the present invention. The specific difference of the dissolution liquid reservoir 100 of the embodiment from that of FIG. 9 resides in that the portion of the diaphragm 102 in contact with the solution is circularly formed in the same manner as in the diaphragm 102 and the protruding portion 103 has a cruciform top end portion 104. Assuming that the lengths of the two bars of the cruciform top end portion 104 are represented by L10 and L11, respectively, and the diameter of the solution contact portion of the diaphragm 102 is represented by L2, they are constructed so as to satisfy the following relationship:
0.1×L2≦L10≦0.5×L2 and/or 0.1×L2≦L11≦0.5×L2.
In this way, when the protruding portion 103 is pressed in use and the diaphragm 102 is broken widely by the cruciform top end portion 104, the solution can flow out well, with the result that the remaining amount of solution can be reduced.

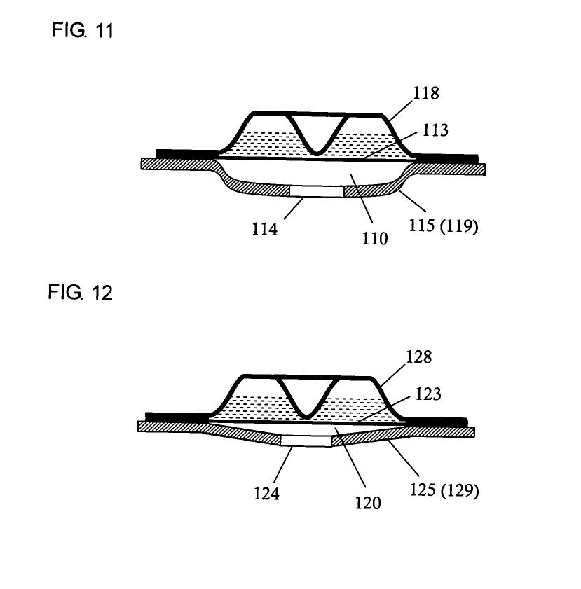

FIG. 11 is a sectional view showing a structure of the support or the liner according to the present invention. In the embodiment, the structure is designed such that the solution is quickly supplied from the dissolution liquid reservoir to an absorber when used. The structure of the embodiment can be applied to the supporting bodies of FIGS. 1 to 3 and FIGS. 7 and 8, and to the liners of FIGS. 4 to 6. In the support 115 or the liner 119 of this embodiment, the portion around the opening 114 through which a solution flows is depressed toward the absorber compared to the other portion, that is, in the opposite side of the dissolution liquid reservoir 118. By virtue of the structure, a space 110 is formed between the support 115 or the liner 119 and the dissolution liquid reservoir 118. When the dissolution liquid reservoir 118 is pressed in use, the diaphragm 113 is broken and a broken piece of the diaphragm can be spread in the space 110, the solution can quickly flows through the opening 114, with the result that the remaining amount of solution can be reduced.

FIG. 12 is a sectional view showing another structure of the support or the liner according to the present invention. Also, in this embodiment, the structure is designed such that the solution stored in the dissolution liquid reservoir is quickly supplied to the absorber in use. The structure of the embodiment can be applied to the supporting bodies of FIGS. 1 to 3 and FIGS. 7 and 8, and to the liners of FIGS. 4 to 6. A support 125 or a liner 129 of the embodiment inclines from the peripheral portion toward an opening 124 with respect to the absorber, as shown in the figure. Also, in this case, a space 120 is formed between the support 125 or the liner 129 and a dissolution liquid reservoir 128. When the dissolution liquid reservoir 128 is pressed in use and the diaphragm 123 is broken, the solution quickly flows through the opening 124 without spreading to the peripheral portion, with the result that the remaining amount of solution in the dissolution liquid reservoir can be reduced.

The dissolution liquid container is formed of the dissolution liquid reservoir in combination with the diaphragm. Now, a preferred structure of the dissolution liquid container will be described below. The dissolution liquid container has a dissolution liquid reservoir having a protruding portion processed to at least a portion. The protruding portion can be formed by molding so as to have a strength capable of breaking the diaphragm by pressure. Therefore, a sheet (film) containing aluminium may not be used for forming the protruding portion since it is easily cracked or broken.

In the present invention, the sheet material (film) for use in the dissolution liquid container (dissolution liquid reservoir) preferably has a water-vapor permeability of 0.30 g/m²·24 hr or less, and more preferably, 0.22 g/m²·24 hr or less. As such a sheet material, for example, a film containing a cyclic polyolefin copolymer (COC) and a fluorocarbon resin may be mentioned. Preferably, a fluorocarbon resin laminate film may be used. Furthermore, if the inner surface of the container and the diaphragm can be sealed with heat, excellent air tightness can be obtained. For this reason, the fluorocarbon resin laminate film is preferably laminated with polyvinyl chloride (PVC), polypropylene (PP) or polyethylene (PE) films. Furthermore, from a drug absorption point of view, a laminate film formed of these films and a polypropylene or polyethylene film is more preferable. The thickness of the sheet material (film) is about 500 μm or less, preferably about 100 to 400 μm, more preferably about 250 to 350 μm, in view of breakability and processability. The diaphragm is preferably formed of aluminium foil.

### EXPERIMENTAL EXAMPLES

### Preparation of the Dissolution Liquid Container

A dissolution liquid reservoir having a protruding portion shown, for example, in FIG. 3, was formed by mold-processing. To the dissolution liquid reservoir, a solution (600 μl of a 30 w/w % aqueous glycerin solution) was added and a processed aluminum foil (20 μm) on which an acrylic adhesive-material (DURO-TAK87-2516, 50 μm) is laminated at 140 to 150° C. for 2 seconds is sealed with heat. In this manner, a dissolution liquid container was formed.

Preparation of a Patch Having a Dissolution Liquid Integrated Therein:

The acrylic adhesive material surface of the dissolution liquid container prepared above was adhered to the upper surface of a preparation to form the preparation shown, for example, in FIG. 3.

Preparation of Patch Having an Alprostadil-Containing Solution Integrated Therein

The acrylic adhesive material surface of the dissolution liquid container prepared above was adhered to the upper surface of a preparation and a drug (alprostadil alfadex 5 mg) and lactose (8 mg) were added to a drug containing layer of the preparation as shown, for example, in FIG. 3, to prepare a preparation. The preparation thus prepared was stored in an aluminium package together with a desiccant (Tri sorb 1 g, manufactured by Sud-Chemie).

Now, Examples and Comparative Examples will be shown below.

### Example 1

The material of a dissolution liquid container (dissolution liquid reservoir) was a laminate of PE film/COC film/PE film (PE/COC/PE). The thickness of the laminate was 350 μm and a water-vapor permeability was 0.22 g/m²·24 hr.

### Example 2

The material of a dissolution liquid container (dissolution liquid reservoir) was a laminate of PP film/PCTFE film (fluorocarbon resin) film (PP/ PCTFE). The thickness of the laminate was 300 μm and a water-vapor permeability was 0.11 g/m²·24 hr.

### Example 3

The material of a dissolution liquid container (dissolution liquid reservoir) was a laminate of PP film/PCTFE film (fluorocarbon resin) film (PP/ PCTFE). The thickness of the laminate was 250 μm and a water-vapor permeability was 0.14 g/m²·24 hr.

### Comparative Example 1

The material of a dissolution liquid container (dissolution liquid reservoir) was a PVC film. The thickness of the film was 300 μm and a water-vapor permeability was 2.70 g/m²·24 hr.

### Comparative Example 2

The material of a dissolution liquid container (dissolution liquid reservoir) was a PP film. The thickness of the film was 300 μm and a water-vapor permeability was 0.70 g/m²·24 hr.

### Comparative Example 3

The material of a dissolution liquid container (dissolution liquid reservoir) was a PP film. The thickness of the film was 500 μm and a water-vapor permeability was 0.32 g/m²·24 hr.

### Comparative Example 4

The material of a dissolution liquid container (dissolution liquid reservoir) was a laminate of PE film/COC film/PE film (PE/COC/PE). The thickness of the laminate was 500 μm and a water-vapor permeability was 0.14 g/m²·24 hr.

### Comparative Example 5

The material of a dissolution liquid container (dissolution liquid reservoir) was a laminate of Al film/PP film (Al/PP). The thickness of the laminate was 150 μm and a water-vapor permeability was 0 g/m²·24 hr.

The Examples and Comparative Examples were evaluated for the processability of the protruding portion of the dissolution liquid container and the breakability of the protruding portion through the diaphragm. The results shown in Table 1 were obtained.

TABLE 1
The material of container and properties

| | | | | Process- | |
|---|---|---|---|---|---|
| | Material | | Water-vapor | ability of | Break- |
| | of | Thickness | permeability | protruding | ability of |
| | container | (μm) | (g/m²) | portion | diaphragm |
| Comparative | PVC | 300 | 2.70 | ∘ | ∘ |
| Example 1 | | | | | |
| Comparative | PP | 300 | 0.70 | ∘ | ∘ |
| Example 2 | | | | | |
| Comparative | PP | 500 | 0.32 | ∘ | x |
| Example 3 | | | | | |
| Comparative | PE/COC/PE | 500 | 0.14 | ∘ | x |
| Example 4 | | | | | |
| Comparative | Al/PP | 150 | 0 | x | — |
| Example 5 | | | | | |
| Example 1 | PE/COC/PE | 350 | 0.22 | ∘ | ∘ |
| Example 2 | PP/PCTFE | 300 | 0.11 | ∘ | ∘ |
| Example 3 | PP/PCTFE | 250 | 0.14 | ∘ | ∘ |

Water-vapor permeability: 40° C., 90% RH, 24 hr (24 hours)

As shown in Table 1, in Examples 1 to 3, Comparative Examples 1 and 2, the protruding portion exhibited good processability and the diaphragm had good breakability (symbol: O). In Comparative Examples 3 and 4 (film thickness of about 500 μm), the protruding portion exhibited good processability (symbol: O); however, the diaphragm had extremely poor breakability (symbol: x) since the strength of the dissolution liquid container is high. In Comparative Example 5, breakage of aluminium was observed in the protruding portion during processing, and thus the processability of the protruding portion was poor (symbol: x).

Next, the weight change of the dissolution liquid container and deliver rate of the solution were evaluated depending upon temperature conditions. A patch integrated with a solution was prepared and stored for a month at temperatures of 40 and 50° C., separately. The reduced amount of the solution (by percentage based on the initial amount) and the deliver rate of the solution were evaluated. The results shown in Table 2 were obtained. The deliver rate of the solution was evaluated based on the time required for delivering of the solution over the entire surface of the drug holding film after the dissolution liquid container was pressed. That is, if the time is within about 30 seconds, symbol O was given and if the time is about 30 seconds or more, symbol x was given.

TABLE 2
Weight change and deliver rate of the solution
depending upon temperature conditions

| | | 40° C., one month | | 50° C., one month | |
|---|---|---|---|---|---|
| | Material | Reduction | Deliver | Reduction | Deliver |
| | of | rate of | rate of | rate of | rate of |
| | container | solution | solution | solution | solution |
| Comparative | PVC | 15.3% | ∘ | 27.6% | x |
| Example 1 | | | | | |
| Comparative | PP | 5.9% | ∘ | 14.1% | ∘ |
| Example 2 | | | | | |
| Example 1 | PE/COC/PE | 2.0% | ∘ | 5.6% | ∘ |
| Example 2 | PP/PCTFE | 0.3% | ∘ | 1.0% | ∘ |

The reduction rate of the solution suitable for long term storage is desirably 13% or less. The solutions of Examples 1 and 2 are suitable for long-term storage, since they exhibited reduction rates of not more than 13% at temperatures of 40° C. and 50° C. On the other hand, significant reduction rates of the solution were observed at temperatures of 40° C. and 50° C. in Comparative Example 1 and at a temperature of 50° C. in Comparative Example 2. They are not suitable for long-term storage. Furthermore, the time required for delivering the solution to a drug holding film for activating a drug in use was short in Example 1 and 2 and Comparative Example 2; however, long in Comparative Example 1.

When the results of Tables 1 and 2 are generalized, as shown in Examples 1 to 3, the sheet material (film) used in a dissolution liquid container (reservoir) preferably has a water vapor permeability of 0.22 g/m²·24 hr or less and a thickness of about 250 μm to about 350 μm. Such a sheet contains a cyclic polyolefin copolymer (COC) film and a fluorocarbon resin film, and is preferably a laminate of a cyclic polyolefin copolymer (COC) film and fluorocarbon resin film or a laminate of a fluorocarbon resin film and polyolefin film.

### INDUSTRIAL APPLICABILITY

The present invention is applicable to a patch for external use in the medical field. The present invention made it possible to provide a patch activated in use, having a dissolution liquid reservoir, being capable of maintaining the stability of a drug, excellent in general use and practical use, and easily applicable. Furthermore, in the patch according to the present invention, since less amount of the solution remains in the dissolution liquid reservoir when used, the drug contained in the patch can be ensured at a predetermined content.

1. A patch activated in use comprising: an absorber containing a dry drug and formed of a material capable of absorbing a liquid; a wall material arranged around the absorber and having an adhesive layer on the lower surface thereof; a support arranged on the absorber and the wall material and having an opening at the center; a diaphragm arranged on the support; and a dissolution liquid reservoir arranged on the diaphragm, holding a dissolution liquid dissolving the drug in a space with the diaphragm, and having a protruding portion which breaks the diaphragm by pressure.

2. The patch activated in use according to claim 1, further comprising a solution permeable film on the lower surface of the absorber.

3. The patch activated in use according to claim 1, further comprising a liner having a concaved portion facing the absorber on the lower surface of the absorber and the adhesive layer.

4. A patch activated in use comprising: a drug containing layer containing a dry drug; an absorber arranged on the drug containing layer and formed of a material capable of absorbing a liquid; a wall material arranged around the absorber and having an adhesive layer on the lower surface thereof; a support arranged on the absorber and the wall material and having an opening at the center; a diaphragm arranged on the support; and a dissolution liquid reservoir arranged on the diaphragm, holding a dissolution liquid dissolving the drug in a space with the diaphragm, and having a protruding portion which breaks the diaphragm by pressure.

5. The patch activated in use according to claim 4, further comprising a liner having a concaved portion facing the drug containing layer on the lower surface of the drug containing layer and the adhesive layer.

6. A patch activated in use comprising: a support; an absorber arranged on the support, containing a dry drug, and formed of a material capable of absorbing a liquid; a wall material arranged on the support and around the absorber, and having an adhesive layer on the upper surface thereof; a liner arranged on the absorber and the adhesive layer and having an opening at the center; a diaphragm arranged on the liner; and a dissolution liquid reservoir arranged on the diaphragm, holding a dissolution liquid dissolving the drug in a space with the diaphragm, and having a protruding portion which breaks the diaphragm by pressure.

7. The patch activated in use according to claim 6, further comprising a solution permeating film on the upper surface of the absorber.

8. A patch activated in use comprising: a support; an absorber arranged on the support and formed of a material capable of absorbing a liquid; a wall material arranged on the support and around the absorber, and having an adhesive layer on the upper surface thereof; a drug containing layer arranged on the absorber and containing a dry drug; a liner arranged on the drug containing layer and the adhesive layer, and having an opening at the center; a diaphragm arranged on the liner; and a dissolution liquid reservoir arranged on the diaphragm, holding a dissolution liquid dissolving the drug in a space with the diaphragm, and having a protruding portion which breaks the diaphragm by pressure.

9. The patch activated in use according to claim 1, wherein a portion of the diaphragm in contact with the dissolution liquid has an oval shape and the protruding portion of the dissolution liquid reservoir has a linear top end portion extending along the longitudinal axis of the oval.

10. The patch activated in use according to claim 9, wherein assuming that the length of the linear top end portion is represented by L1 and the length of the longitudinal axis of the portion of the diaphragm in contact with the dissolution liquid is represented by L2, the following relationship is satisfied.
0.1×L2≦L1≦0.5×L2

11. The patch activated in use according to claim 1, wherein a portion of the diaphragm in contact with the dissolution liquid has a circular shape and the protruding portion of the dissolution liquid reservoir has a cruciform top end portion.

12. The patch activated in use according to claim 10, wherein assuming that the lengths of both bars of the cruciform top end portion are represented by L10 and L11, respectively, and the diameter of the portion of the diaphragm in contact with the dissolution liquid is represented by L2, the following relationship is satisfied.
0.1×L2≦L10≦0.5×L2 and/or 0.1×L2≦L11≦0.5×L2

13. The patch activated in use according to claim 1, wherein the portion of the support around the opening is depressed toward the absorber compared to the other portion.

14. The patch activated in use according to claim 1, wherein the support inclines from the peripheral portion toward the opening with respect to the absorber.

15. The patch activated in use according to claim 6, wherein the portion of the liner around the opening is depressed toward the absorber compared to the other portion.

16. The patch activated in use according to claim 6, wherein the liner inclines from the peripheral portion toward the opening with respect to the absorber.

17. The patch activated in use according to claim 1, wherein the dissolution liquid reservoir is formed by subjecting a sheet material to mold processing and the sheet material has a water vapor permeability of 0.22 g/m²·24 hr or less.

18. The patch activated in use according to claim 17, wherein the sheet material has a thickness of about 250 μm to about 350 μm.

19. The patch activated in use according to claim 17, wherein the sheet material contains a cyclic polyolefin copolymer film.

20. The patch activated in use according to claim 17, wherein the sheet material is a laminate film of a cyclic polyolefin copolymer film and a polyolefin film.

21. The patch activated in use according to claim 17, wherein the sheet material includes a fluorocarbon resin film.

22. The patch activated in use according to claim 17, wherein the sheet material is a laminate film of a fluorocarbon resin film and a polyolefin film.

23. The patch activated in use according to claim 1, wherein the diaphragm is an aluminium foil.
